(19) United States
(12) Patent Application Publication
(10) Pub. No.: US 2024/0367489 A1
(43) Pub. Date: Nov. 7, 2024

(54) COLORED AUTOMOTIVE WINDOWS

(71) Applicants: Scott FREEMAN, Las Vegas, NV (US); Sir KINGSLEY, Las Vegas, NV (US)

(72) Inventors: Scott FREEMAN, Las Vegas, NV (US); Sir KINGSLEY, Las Vegas, NV (US)

(21) Appl. No.: 18/174,840

(22) PCT Filed: Sep. 14, 2021
(86) PCT No.: PCT/US2021/050176

§ 371 Date: Feb. 27, 2023

Related U.S. Application Data
(60) Provisional application No. 63/078,078, filed on Sep. 14, 2020.

(51) Int. Cl. B60J 1/00; B32B 17/10

(52) U.S. Cl. CPC B60J 1/001; B32B 17/10036; B32B 17/10651; B32B 17/10899; B32B 2250/03; B32B 2250/40; B32B 2307/4026; B32B 2605/00

(57) ABSTRACT

A vehicle including at least one colored automotive glass window operatively attached to the vehicle. A method of providing colored automotive glass windows in a vehicle, by making a colored automotive glass window by a method of 1) adding a metal compound or other chemical to molten glass during the manufacturing process forming colored glass, 2) by bonding multiple layers of glass together while under pressure and heat, with a colored interlayer to make colored laminated glass, and 3) by layering a colored plastic, chemical, or compound on top of a clear glass window, and installing the colored automotive glass window in a vehicle. A method of providing health benefits to an individual by the individual being inside or outside a vehicle with at least one colored automotive glass window operatively attached thereto, and the at least one colored automotive glass window providing health benefits to the individual.

### BACKGROUND OF THE INVENTION

### 1. Technical Field

The present invention relates to compositions and methods for providing colored glass windows for automobiles to improve health and safety.

### 2. Background Art

The average American spends 18 days a year in their vehicle, which corresponds to approximately 8 hours a week. This average is higher in large cities like Los Angeles where traffic congestion causes a person to spend an additional 100 hours a year in their car. Cars are a necessity for most people, particularly in rural areas or large urban cities with poor mass transit, and therefore, a person often must spend this much time in their car. The safety, well being, and comfort of a vehicle is very important for the person(s) in the automobile since so much time is spent in one. It is stressful driving a car for several reasons: 1) operating a motor vehicle requires concentration since traveling at any speed particularly speeds above 50 mph can lead to injury and death if a driver is not alert. Even at low speeds automobiles can be deadly; many accidents and injuries occur within a few minutes of home at slower speeds on local roads. 2) There is often congestion on the roadways and “sitting in traffic” can be very stressful even if the motor vehicle is moving at a slow speed. Couple the stress of driving with normal daily activities and a person with a mental health issue or on the verge of one can be tipped into a worse state by spending so much time in an automobile.

Making an automobile safer and providing health benefits would alleviate both the stressors of driving a car and also improve the overall mental health and well being of person(s) in the vehicle and this invention does both.

In addition, making an automobile feel “homier” by aesthetics gives a person a sense of control and comfort, like decorating a house. This in and of itself can alleviate stress.

No invention to date has used colored glass windows in an automobile driven on a public road or highway for safety and health benefits.

Since the average person drives 400-500 hours a year and sees hundreds of thousands of cars a year on the public roads and highways if they drive in a large city, by the time a person is 40 years old they have driven about 10,000 hours and have seen millions of cars, yet virtually no person has ever seen a car with a colored glass window on a public road or highway even though cars have been in existence for over 100 years.

Cars have clear glass windows because a function of a car glass window is for the person inside the car to see outside the car. This is different than for instance a glass window in a building where privacy can be important and window shades, blinds, and colored glass windows are constructed to prevent people outside the building from looking in. Intuitively most people believe a clear glass window is best for car windows because it provides the best visualization and it is counterintuitive to think colored glass windows can meet visual needs and thus colored glass windows for cars have never been invented, but colored glass windows can improve visibility in certain conditions by reducing glare and eye strain and improving depth perception to name a few. Thus, colored glass windows can perform the visibility function of a car window while also providing a safety and health benefit for persons inside and a health benefit for persons outside the car, as described below.

Virtually all automobile windows are made of clear glass and the only colored car windows can be observed in a special venue like a hot rod racing event or an automobile show. There are several reasons: 1) car windows are for seeing out of the vehicle and it is counterintuitive to add color since the average person considers a clear window to provide better visibility than a colored window which is assumed to have less visual acuity than a clear window and 2) adding colored windows to cars would add a complexity to car sales and marketing that automobile makers do not want, e.g., another option for buyers and thus increasing inventory.

The assumption that automotive glass windows should be clear to make them highly visible is highlighted in U.S. Pat. No. 9,012,015, “It should be noted that it is preferable that the laminated glass with the plastic film insert according to the present invention can be used for the window which is needed for the driving of the automotive vehicle by making the visible light transmittance prescribed in JISR3212:1998 equal to or larger than 70%.” This highlights that the current thinking in the auto industry is that 70% light transmission, which corresponds to a light color shade, offers the best visibility and all states in the United States requires that at least one window has 70% light transmission. However, most cars have 6 windows, a front windshield, a back windshield, two side front windows, and two side back windows and there are different regulations for light transmission of each window depending on location, which can vary from 0% to 70% light transmission depending on the location of the window in the car and US State or country regulations.

To date, almost all automobiles with colored windows, albeit very few, have been found in specific settings like hot rod racing venues or automotive shows. The average person has never seen a car on a public road or highway with colored glass windows even though they observe hundred of thousands of cars a year on the public roads and highways if they drive in a large city like Los Angeles, Dallas, Chicago, San Francisco, and New York. This makes sense since the car windows are made to see out of the car and the best view is thought to be with clear glass windows. Colored glass windows are usually used in buildings and room walls for privacy to prevent an outside person from looking in or to prevent sun rays (UV light and heat) or sound from entering the building or room or for aesthetic purposes. Automobiles have been in use for over 100 years yet although colored body parts are used, glass automotive windows are clear because it has been counterintuitive, and not thought about, to believe that colored glass windows can provide both adequate vision and a health benefit.

Color therapy or Chromotherapy or photobiomodulation (PBMT) has been in existence for millenniums. Chakra meaning “color wheel” was first mentioned in Hindu texts in 1^{st} millennium BCE and later appeared in the Buddhist text around the 8^{th} century CE. The Chakras are part of medieval-era beliefs about physiology and psychic centers that emerged across Indian traditions. There are seven Chakra for energy, and each corresponds to a visible color of the light spectrum.

Red is balance and activates the root Chakra associated with passion, sexual energy, and reconnection with mother earth. Orange removes blockages from sacral Chakra for joy and contentment. Yellow removes blocks from solar plexus Chakra for clarity of mind. Green evokes feelings of love, harmony and peace. Blue is throat Chakra removing problems in communication. Indigo is associated brow Chakra and deals with intuition and sixth sense. Violet is linked to the crown Chakra of higher consciousness.

It makes sense that light forms, sunlight and colored light, can affect a person's well being. The health benefits of sunlight are well known from the fact that sunlight is required for the body to produce vitamin D. Persons in low light areas develop a disease termed Ricketts from lack of vitamin D. Light therapy was also found to help premature newborns with jaundice, high bilirubin. Exposure to light decreases bilirubin and blue light therapy is the most effective form of color therapy for jaundice. Blue light can also have its effects on the brain with blue light from computers and cell phones affecting the circadian rhythm, the sleep-wake cycle, by decreasing the amount of melatonin produced by the pineal gland in the brain, which particularly at night prevents the onset of sleep. Hence chromotherapy or biophotomodulation, colored light, has been shown to affect human health.

There are three ways colored light through chromotherapy can affect human health: 1) the visual light pathway, seeing color for mood and mental health, 2) the non-visual pathway where optic neurons from the eye are activated by a specific color of light whereby some neurons signal through the optic cortex neurons (visual) while other neurons send signals to other parts of the brain like the hypothalamus (non-visual), as described above so that blue light can affect the release of melatonin, and 3) a direct effect of light on organs, like blue light on the skin. An example is a baby with jaundice where the blue light helps conjugate bilirubin for excretion in the bile.

Motor vehicles are used outdoors and can be parked outdoors thus exposing them to weathering elements like sunlight which exposes the inside of the car to heat and UV light thus affecting the “health” of the car. Colored glass windows can be made to block UV light, heat transmission from sunlight, and sound, thus “protecting” the interior of the automobile.

Since a person spends so much time in their automobile, their physical and emotional stability may be affected. New methods need to be developed to help offset the physical and mental changes caused by being in a vehicle for long periods of time during the year. A person's mood and surrounding peoples' moods can be affected by color and thus the color of a vehicle glass windows can affect mood. The person in the car and persons looking at the car can have their mood improved by a car's colored windows in several ways. Colored windows, depending on the color can alter a person's mood: happiness, cheerfulness, contemplative, thoughtful, calmness, to name a few.

Brightly colored windows can affect a person's cheerfulness especially during winter months in places where sunlight is less and daylight hours are short like Canada, Northern United States, Scandinavian countries, Iceland and the like.

Imagine visiting a garden with brightly colored flowers in bloom and the mood enhancing effects of that experience. Similarly, a highway with thousands of cars with different colored windows will have a similar effect like that of a flower garden on a person in another car observing all the colored car windows passing by. Thus, not only can a person's mood be enhanced by putting colored windows in their own car which can improve their mood and well being by visualizing the color and by “decorating” their car in a color of choice, like decorating a home, but also by observing other cars on the public road with colored windows.

Each color can have a different effect on a person as shown in the color symbolism chart in FIG. 1.

A color can have both a positive and negative effect and thus colors become a very personal choice. For instance, orange's positive effects are spontaneity, creativity, warmth, positivity, while the negative effects are exhibitionism, superficiality, impatience, and domination. Red can have positive effects on sexuality, courage, desire, and confidence and negative on anger, danger, revenge and aggression, while green is positive on generosity, hope, prosperity, luck and negative on judgmental, envy, materialism, inexperience. This positive and negative effect can be setting related. For instance, studies have shown that showing a student a red card prior to taking a test leads to a worse test score than students shown a white card (Elliot AJ, J exp Psychol, General 136 (1) 154, 2007).

Red and yellow have also been shown to increase heart rate while blue causes relaxation in a student learning environment.

In addition, a person sitting in the car can receive a therapeutic benefit from different colored lights touching their body, termed chromotherapy or photobiomodulation. Chromotherapy is a method of treatment that uses the visible spectrum (colors) of electromagnetic radiation to cure diseases. It is a centuries-old concept used successfully over the years to cure various diseases. Light is electromagnetic radiation, which is the fluctuation of electric and magnetic fields in nature. More simply, light is energy, and the phenomenon of color is a product of the interaction of energy and matter. The wavelength, frequency, and quantity of energy of every colorful ray are fixed for each color; that is, a specific wavelength, a certain frequency and a particular amount of energy in that wave have been denominated as a distinct color. The human eye is sensitive to electromagnetic radiation only at wavelengths roughly between 380 and 780 nm. This small segment is called the visible spectrum or visible light. Visible light can be broken down into numerous electromagnetic frequencies, and frequency relates to a color of the rainbow: red, orange, yellow, green, blue, indigo, violet and all the vibrations thereof. The key to understanding chromotherapy/vibrational healing lies in rerouting energy fields that form complex relationships with other fields such as those surrounding the physical/cellular substance and others relating to more non-physical energies. Every creature is engulfed in light that affects its health conditions. The human body, according to the doctrine of chromotherapy, is basically composed of colors. Chromotherapy is a narrow band in the cosmic electromagnetic energy spectrum, known to humankind as the visible color spectrum. It is composed of reds, greens, blues and their combined derivatives, producing the perceivable colors that fall between the ultraviolet and the infrared ranges of energy or vibrations. These visual colors, with their unique wavelength and oscillations, applied to impaired organs or life systems, provide the necessary healing energy required by the body.

This concept has a scientific basis in that there are chromophores and porphyrins in the body which can be activated by specific colored lights. Cytochrome oxidase C in mitochondria is one such and since mitochondria are the power source of the cell, light therapy can target this important cell organelle.

Colors generate electrical impulses and magnetic currents or fields of energy that are prime activators of the biochemical and hormonal processes in the human body, the stimulants or sedatives necessary to balance the entire system and its organs. Thus, chromotherapy can work at the cellular level.

During the 1950s, studies suggested that neonatal jaundice, a potentially fatal condition found in two-thirds of premature babies, could be successfully treated by exposure to sunlight. This was confirmed in the 1960s, and white light replaced high-risk blood transfusions in the treatment of this condition. Blue light was later found to be more effective and less hazardous than full-spectrum light.

Chromotherapy is also now being used in the treatment of cancers, SAD (seasonal affective disorder, so-called winter depression), anorexia, bulimia nervosa, insomnia, jetlag, shift working, alcohol and drug dependency, and to reduce overall levels of medication. Some chromotherapy has received FDA approval as described below.

Visible light can be broken down into a rainbow of colors; Red wavelength 700 nanometers, Orange wavelength 600 nanometers, yellow wavelength 580 nanometer, green wavelength 550 nanometers, blue wavelength 475 nanometers, indigo wavelength 450 nanometers and violet wavelength 400 nanometers.

Schauss worked on the tranquilizing effect of colors and found that color reduces aggressive behavior and violence. The blue light found to be successful in the treatment of neonatal jaundice has also been shown to be effective in the treatment of rheumatoid arthritis, as emphasized by Pleasanton in his work. In studies by McDonald, most of those exposed to blue light for variable periods of up to 15 minutes experienced a significant degree of pain relief. It was concluded that the pain reduction was directly related both to the blue light and to the length of exposure to it. Blue light is also used in healing injured tissue and preventing scar tissue, as well as for burns and lung conditions. In 1990, scientists reported to the annual conference of the American Association for the Advancement of Science on the successful use of blue light in the treatment of a wide variety of psychological problems, including addictions, eating disorders and depression. At the other end of the color spectrum, red light has been shown to be effective in the treatment of cancer and constipation and in healing wounds. This may be due to the fact that cells have porphyrins and chromophins. As a result, color is becoming widely accepted as a therapeutic tool with various medical applications. Research also confirmed that certain parts of the brain are light sensitive and respond differently to different wavelengths; it is now believed that different wavelengths (colors) of radiation interact differently with the endocrine system to stimulate or reduce hormone production.

Thus chromotherapy can provide health benefits in several ways: 1) A direct effect of light hitting the skin like light causing production of vitamin D or blue light treating jaundice; 2) Light directly acting on the eye and nerves stimulate the visual cortex, like the enhanced mood effects of seeing red, yellow, or orange; 3) Light activating cells in the retina which can stimulate the nerves in other brain areas, like blue light causing the pineal gland to produce less melatonin affecting the sleep-wake cycle.

This work has given a new dimension to chromotherapy: the use of colors in psychological disorders. Seasonal Affective Disorder (SAD) has become a very common problem nowadays, in England in particular, where the sun does not shine for up to 1 or 2 weeks, so that no light enters into the body. As a consequence, psychological diseases manifest, mainly in the form of depressions, which, according to some chromopathy studies, are curable without any use of tranquilizers.

A detailed study of chromotherapy, with patients exposed to sunlight through color filters, was produced by Dr. Jacob Lieberman, an optometrist. He adopted modern theories to prove the relationship between melatonin, light, and color. Takkata was the first researcher to attempt to find a relationship between blood and sunlight. Dr. Lieberman's work concerns hormonal changes as a result of exposure to sunlight. Of course, sunlight is a perfect blend of seven colors; different colors are responsible for the release of different kinds of hormones, which keeps us healthy. Dr. Jacob Lieberman stated in his work that light is responsible for turning on the brain and the body. Light enters the body through the eyes and skin. When even a single photon of light enters the eye, it lights up the brain. This light triggers the hypothalamus, which regulates all life-sustaining bodily functions, the autonomic nervous system, endocrine system, and the pituitary (the body's master gland). The hypothalamus is also responsible for our body's biological clock through the pineal organ, which is responsible for releasing one of our most important hormones, melatonin. The release of melatonin is directly related to light and darkness, and manages our sleep-wake cycle, circadian rhythm. This necessary hormone affects every cell in the body. It turns on each cell's internal activities, allowing them to harmonize with each other and nature.

As discussed above, chromotherapy is supported by biological experiments. Mitochondria are cell organelles involved in metabolism and energy production. The mitochondria have chromophobes in their membranes like cytochrome c oxidase and other compounds with iron-sulfur centers, flavins, and hemoglobin. These molecules when activated can increase reactive oxygen species (ROS), nitrous oxide (NO), ATP, cAMP, NADPH which can downregulate inflammatory cytokines like IL-6, IL-8, and IL-1 and upregulate epidermal growth factor, transforming growth factor beta and energy compounds in the cell like NADH, ATP, ADP; it's easy to see how wound healing and collagen production can be promoted by chromotherapy or photobiomodulation (PBMT).

There are animal and human studies that demonstrate the effects of photobiomodulation therapy (PBMT). PBMT has also been shown to be an effective treatment of the inflammatory processes in an animal model. Zigmond evaluated the effect of PBMT on mucosal healing in an acute colitis model in mice. PBMT was applied to the colon utilizing a small-diameter endoscope with an LED-based light source at wavelengths of 440, 660, and 850 nm. PBMT commenced 1 day before induction of colitis and continued during the 6-day induction period, as well as for 3-10 days thereafter. Disease activity was scored endoscopically and by histopathological assessment. A statistically significant improvement in disease severity was observed in all 3 PBMT treatment groups (Glass, GE, Aesthet Surg J, 41(6):723-38, 2021).

Neuman and Finkelstein evaluated 660 nm PBMT on the nasal clinical symptoms of allergic rhinitis in a double-blinded randomized prospective study. A statistically significant improvement of symptoms was reported by 72% of the allergic rhinitis patients and objective improvement was endoscopically demonstrated in 70% of the patients treated by narrow-band red light illumination of the nasal mucosa at 660 nm when compared with the placebo group, with marked alleviation of clinical symptoms.

Rigby showed that a combination of blue light and red light therapy decreased muscle fatigue during strength training in 30% of participants and other studies have demonstrated that PBMT can decrease pain.

Using colored glass in windows can provide health benefits through color visualization or the shining of colored light on the person sitting in the car. There are several methods to make colored automotive glass windows. Colored automotive windows can be provided 1) by applying a colored film on top of the clear glass window, 2) by embedding or inserting a colored element like a chemical or metal compound directly in the glass during its making or 3) by putting an interlayer of color, using for instance PVB, between two sheets of glass (laminated glass).

Bob's Classic Auto Glass advertises a few glass colors such as green, gray, or bronze that are mixed directly into the glass. These colors, green, gray, and bronze, seem to be easily available as these colors are used to color glass bottles but the colors are limited to these three colors and have limited use. Grey only comes in a glass window with only 44% light transmission and therefore can only be used in most cases in rear windows to meet US State regulations. The bronze glass windows cannot be curved which is often needed in modern day glass windows for automobiles. It is also not clear if the glass meets safety standards. There are clear problems with Bob's classic colored windows which include: limited colors, limited use because of low light transmission, unable to bend the glass, and unknown safety. In addition, Bob's classic colors don't appear to meet the higher window glass standard as tempered and laminated glass currently used in glass windows. These glass types are important for safety since they don't easily break into pieces upon impact. Bob's classic colors are not available in all possible colors for automobile windows, colors which are needed for health benefits like orange, red, yellow, and blue.

Any colored piece of glass can work for a car window provided it meets the necessary requirements (light transmission) and regulatory standards (DOT testing).

There are two main types of clear glass currently used in car windows because they provide additional safety in that they don't easily shatter into piece when broken. These are: tempered and laminated glass.

Tempered glass, also known as toughened glass, in general, is created by heating and rapidly cooling a pre-cut standard piece of glass in a tempering furnace. The pre-cut and edged piece of glass is heated up to around 1200° F. After being heated up; the glass is then cooled rapidly. This process is known as quenching. Quenching produces a hardened piece of glass that is four to five times stronger than before the tempering process. The final product tempered glass is harder to break. Tempered glass is most commonly used for passenger windows on cars while laminated glass makes up front and rear windshields most of the time. When tempered glass breaks, it is designed to shatter into small pieces that are less likely to cause added injury or damage. Tempered glass can also be treated with chemicals and thermal treatments; these treatments help give the piece of glass more balanced internal stress capabilities. For tempered glass, color needs to be added to the glass during the glass making process to produce color in the glass.

Laminated glass is created by bonding multiple layers of glass together while under pressure and heat, with a resin like polyvinyl butyral (PVB) as an interlayer. This process uses single sheets of glass that have an interlayer like PVB that are sandwiched together and thus have multiple layers. The PVB helps keep the glass from breaking apart easily if shattered as a safety feature and can produce sound insulation and UV light reflectance/absorbance particularly if it's a multi-layered interlayer. Before shattering, laminated glass will bend and flex. Despite not being as strong as tempered glass, laminated glass does not break into pieces but adheres together when broken because of the interlayer, and it also can be made to block ultraviolet light transmission as well as sound and heat transmission with appropriate interlayers. Blocking UV light, sound, and heat would be beneficial to the person sitting in the car. The non-shattering is a safety feature if the glass breaks and also helps prevent vandalism since thieves can't easily and quickly smash a window and steal the inside contents of an automobile and make a quick getaway. Laminated glass has an interlayer sheet of color that holds two pieces of clear glass together. This interlayer can be a substance like polyvinyl butyrate (PVB) between two glass panes.

There are other types of glass that can be used for car windows like annealed glass, chemically strengthened glass, and heat strengthened glass which need to be colored during the glass making process. However, there are other ways to color glass windows after the clear glass windows have been made, like applying a colored film strip onto any of the five types of glass that could be used for car glass windows including tempered glass, laminated glass, annealed glass, chemically and heat strengthened glass.

In any situation, colored glass windows in the car must meet regulatory standards. One such regulatory standard determines how much light transmission is required at a minimum depending where the glass window resides on the car. In general, the glass in the front of the car requires more light transmission than the glass on the back of the car so the driver can see what is ahead. Every state in the United States has a different law regarding light transmission in car windows and different countries can have different requirements. TABLE 1 lists window light laws in the U.S.

TABLE 1
All Window Light Transmission Laws by State in the United States

| | Front | Rear | Rear | |
|---|---|---|---|---|
| State | Sides | Sides | Window | Windshield |
| Alabama | 32% | 32% | 32% | 6″ |
| Alaska | 70% | 40% | 30% | 5″ |
| Arizona | 33% | ANY | ANY | AS1 |
| Arkansas | 25% | 25% | 10% | 5″ |
| California | 70% | ANY | ANY | 4″ |
| Colorado | 27% | 27% | 27% | 4″ |
| Connecticut | 35% | 35% | ANY | AS1 |
| Delaware | 70% | ANY | ANY | AS1 |
| District of Columbia | 70% | 50% | 50% | 5″/AS1 |
| Florida | 28% | 15% | 15% | AS1 |
| Georgia | 32% | 32% | 32% | 6″ |
| Hawaii | 32% | 35% | 35% | 70% light |
| Idaho | 35% | 20% | 35% | AS1 |
| Illinois | 35% | 35% | 35% | 6″ |
| Indiana | 30% | 30% | 30% | AS1 |
| Iowa | 70% | ANY | ANY | AS1 |
| Kansas | 35% | 35% | 35% | AS1 |
| Kentucky | 35% | 18% | 18% | AS1 |
| Louisiana | 40% | 25% | 12% | AS1 |
| Maine | 35% | ANY | ANY | 4″ |
| Maryland | 35% | 35% | 35% | 5″ |
| Massachusetts | 35% | 35% | 35% | 6″ |
| Michigan | 4″ | ANY | ANY | 4″ |
| Minnesota | 50% | 50% | 50% | NONE |
| Mississippi | 28% | 28% | 28% | AS1 |
| Missouri | 35% | ANY | ANY | AS1 |
| Montana | 24% | 14% | 14% | AS1 |
| Nebraska | 35% | 20% | 20% | 5″/AS1 |
| Nevada | 35% | ANY | ANY | AS1 |
| New Hampshire | NONE | 35% | 35% | 6″ |
| New Jersey | NONE | ANY | ANY | NONE |
| New Mexico | 20% | 20% | 20% | 5″/AS1 |
| New York | 70% | 70% | ANY | 6″ |
| North Carolina | 35% | 35% | 35% | AS1 |
| North Dakota | 50% | ANY | ANY | <70% |
| Ohio | 50% | ANY | ANY | <70% |
| Oklahoma | 25% | 25% | 25% | 5″/AS1 |
| Oregon | 35% | 35% | 35% | 6″ |
| Pennsylvania | 70% | 70% | 70% | NONE |
| Rhode Island | 70% | 70% | 70% | AS1 |
| South Carolina | 27% | 27% | 27% | AS1 |
| South Dakota | 35% | 20% | 20% | AS1 |
| Tennessee | 35% | 35% | 35% | AS1 |
| Texas | 25% | 25% | ANY | 5″/AS1 |
| Utah | 43% | ANY | ANY | AS1 |
| Vermont | NONE | ANY | 32% | 32% |
| Virginia | 50% | 35% | 35% | AS1 |
| Washington | 24% | 24% | 24% | 6″ |
| West Virginia | 35% | 35% | 35% | 5″ |
| Wisconsin | 50% | 35% | 35% | AS1 |
| Wyoming | 28% | 28% | 28% | 5″/AS1 |

According to the Federal Register from the Department of Transportation (DOT), there are a series of 13 tests for glass to pass in order to comply with FMVSS No. 205. They have derived these tests from the American National Standard for Safety Glazing Materials for Glazing Motor Vehicles and Motor Vehicle Equipment Operating on Land Highways-Safety Standard (ANSI for short). They cover a range of issues including: Radiation (Light Stability) Test, Luminous Transmittance Test, Humidity and High-Temperature Resistance Test, Half-pound Ball Impact Test (Tempered Glass), Fracture Test (Tempered Glass), Shot Bag and Dart Drop Tests (Tempered Glass), Half-pound Ball Drop Test (Laminated Glass), Weather Test (Plastic and Glass-plastic Glazing), Abrasion Resistance Test, Optical Distortion Test (Glazing Materials Used As Windshields), Chemical Resistance, Change in Temperature, and Flammability Tests, Penetration Resistance Test (Laminated Glass), and Optional 10 kg (221b) Headform Drop Test (not currently included in ANSI Z26.1)

The tests simulate and determine the viability of the materials in the event of an accident, disaster, or impact across a variety of scenarios and situations.

Therefore, there remains a need for colored automotive windows in a wide range of different colors, with ranges of light transmission that meet regulatory standards, and safety standards (DOT testing).

### SUMMARY OF THE INVENTION

The present invention provides for colored automotive glass windows for use in an automobile for health, safety, and aesthetics, and more specifically for a vehicle including at least one colored automotive glass window operatively attached to the vehicle.

The present invention provides for a method of providing colored automotive glass windows in a vehicle. The colors can be any color in the visible light spectrum: red, orange, yellow, green, blue, indigo, violet, or combination. Making the colored automotive glass can be done by 1) adding a metal compound or other chemical to molten glass during the manufacturing process forming colored glass of any color, 2) by bonding multiple layers of glass together while under pressure and heat, with a colored interlayer/resin to make laminated glass or 3) by layering a colored plastic, chemical or compound on top of the clear glass window. The colored glass is then installed into the automobile as windows; the colored glass window can replace one or more of the automobiles windows. Depending on where the glass window is installed there can be different regulatory standards for light transmission.

The present invention provides for a method of providing health benefits to an individual, by the individual being inside or outside a vehicle with at least one colored automotive glass window operatively attached thereto, and the at least one colored automotive glass window providing health benefits to the individual.

### DESCRIPTION OF THE DRAWINGS

Other advantages of the present invention are readily appreciated as the same becomes better understood by reference to the following detailed description when considered in connection with the accompanying drawings wherein:

FIG. 1 is a chart showing color symbolism;

FIG. 2 is a photograph of a vehicle including an orange colored automotive glass window;

FIG. 3 is a photograph from inside a vehicle including a colored automotive glass window;

FIG. 4 is a photograph from inside a vehicle including a colored automotive glass window; and

FIG. 5 is a specification sheet for the orange colored interlayer in the laminated glass of the automotive orange glass window.

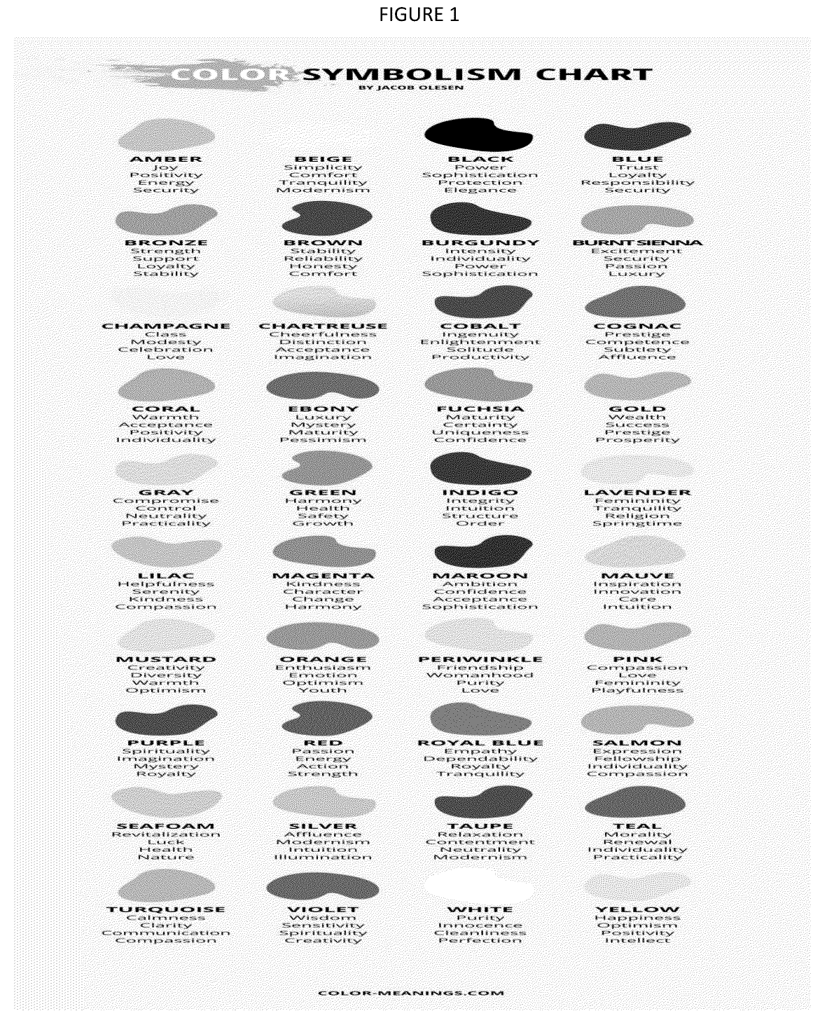

### DETAILED DESCRIPTION OF THE INVENTION

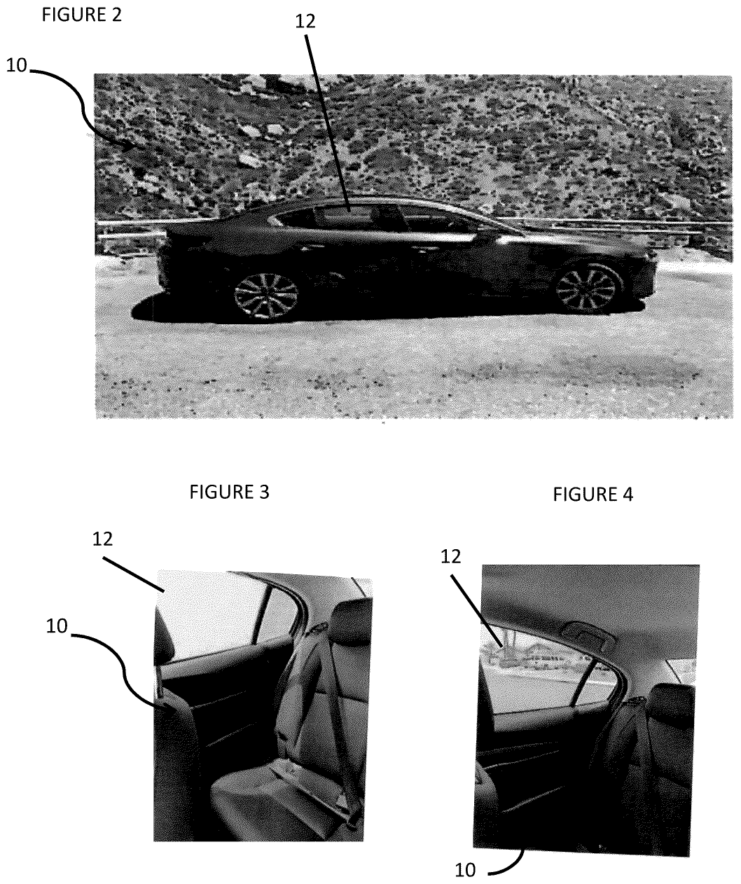

The present invention provides for colored automotive glass for use in a vehicle. The colored automotive glass can be custom colored to suit an individual's eyesight, improve visibility in a particular city's weather, provide health benefits (such as by 1) shining of colored light on the body which is termed chromotherapy or photobiomodulation (PBMT) or 2) visually observing colors), prevent deterioration of some automotive parts, or match the paint color of a vehicle for aesthetics. More specifically, the present invention provides for a vehicle 10 including at least one colored automotive glass window 12 operatively attached thereto, shown in FIGS. 2-4 in an orange color.

The colored automotive glass can be any color in the visible light spectrum: red, orange, yellow, green, blue, indigo, violet or a combination of colors. The colored glass window can also have at least two different colors such as light orange on the top half and red on the bottom half. Different colors provide different advantages. Green color provides contrast by filtering out blue light, reducing glare and eye strain in bright sunlight. Gray color reduces glare from bright light, works on both cloudy and sunny days, and provides anti-fatigue benefits. Blue or purple colors provide enhanced color perception to the individual, help in seeing contours around objects more clearly while offering protection from reflective surfaces (such as snow), and work well in foggy and misty weather. Red or pink colors block blue light and improve driving visibility while reducing eye strain and help increase depth of field and details. Yellow, orange, or gold colors can be used in moderate to low-level light conditions, provide good depth perception at night, improve visibility of objects and make surroundings appear brighter and block out blue light.

Bright colors like red and yellow can help filter out UV light which has increased with global warming. Although automobile glass has some UV protection, driving in sunlight, depending on the angle of the sun, can lead to sunburns on the arms, legs, and face that are exposed to the sun. Thus, colored glass can add additional protection to current automotive glass. UV light has been increasing with global warming and colored windows can block the harmful UV rays from the sun and help prevent skin cancer. Research shows that 53% of skin cancers in the U.S. occur on the left side of the body, which correlates to the driver's side of a car. For people who drive for extended periods of time, colored windows provide a critical advantage to safeguard against this risk. This is especially important for individuals who spend a lot of time in their vehicle in sunny locations. Colored windows can also help reduce the amount of solar heat generated in the vehicle by up to 70%, which reduces the use of air conditioning, saves fuel consumption, and prevents fading and destruction of the interior. Colored windows can also reduce sun damage to the interior of the vehicle from fading upholstery or discoloring leather and vinyl.

UVB rays have slightly longer wavelengths (280-315 nm) and lower energy than UVC rays which are mostly filtered out by ozone layer, but some still reach the Earth's surface. In low doses, UVB radiation stimulates the production of melanin (a skin pigment), causing the skin to darken, creating a suntan. But in higher doses, UVB rays cause sunburn that increases the risk of skin cancer. UVB rays also cause skin discolorations, wrinkles and other signs of premature aging of the skin. Overexposure to the sun's UVB radiation also is associated with a number of eye problems, including pinguecula, pterygium, and photokeratitis (“snow blindness”). Red and blue are better at blocking UVB than yellow.

UVA light can pass through the cornea and affect the retina causing macular degeneration.

Children are more susceptible to UV exposure. But UV exposure also increases for adults with altitude, closeness to the equator, time of day when sun is highest in the sky (10 AM to 2 PM), and settings like wide open spaces and exposure to reflective surfaces. Also, medications such as tetracycline, sulfa drugs, birth control pills, diuretics and tranquilizers, can increase the body's sensitivity to UV radiation. Persons, depending on a multitude of circumstances, can add extra protection from UV exposure in their vehicle by using colored glass windows.

Colored glass automotive windows can affect a person's mood and mental health. Red, yellow and green colors are cheerier and can help prevent depression providing a lasting health benefit since a person spends 18 days a year in a car, an amount of time that a person may take for vacation during the year. And since automobile travel is a necessity for many people and cannot be easily avoided, mental health can be an issue and colored windows can help improve mental health and prevent depression.

Red, orange and yellow are next to each other on the color wheel and are all warm colors. Warm colors often evoke feelings of happiness, optimism and energy. However, yellow, red and orange can also have an attention grabbing effect and signal danger or make you take action (used in stop signs, hazard warnings and barrier tape). Red can also increase a person's appetite.

Cool colors include green, blue, and purple. Cool colors are usually calming and soothing but can also express sadness. Purple is often used to help spark creativity as it is a mixture of blue (calm) and red (intense). If a company wants to display health, beauty or security, they can incorporate these colors.

Happy colors are bright, warm colors like yellow, orange, pink and red. Pastel colors like peach, light pink, or lilac can also have an uplifting effect on mood. The brighter and lighter a color, the happier and more optimistic it will make one feel. Another way colors can create happy emotions is by combining multiple primary and secondary colors together for a youthful, colorful effect.

Sad colors are colors that are dark and muted. Grey is the quintessential sad color, but dark and muted cool colors like blue, green or neutrals like brown or beige can have a similar effect on feelings and emotions depending on how they are used.

Cool colors like blue and green can make you feel calm. Pastel colors and particularly cool toned pastels like baby blue, lilac and mint have a calming and relaxing effect. Neutrals like white, beige and grey can also make one feel calm. The fewer colors combined and the simpler and more pared back a design is, the more calming it is.

Strong, bright colors can have a powerful effect on emotions. Colors like bright red, bright yellow, and neon green can feel energizing and make one feel more alert. These colors will grab attention and stand out from their surroundings. Highly pigmented, strong colors like royal blue, turquoise, magenta and emerald green can also have a stimulating effect and make one feel refreshed and energized.

Chromotherapy, the shining of light on the body, has been used for centuries and studied using modern scientific methods since in the 20th century. “Chromotherapy” is sometimes used interchangeably with both “light therapy” and “color therapy” and “photobiomodulation (PBMT)”, causing some confusion. (Note that “color therapy” is a term typically used to describe the design of spaces, such as hospitals and schools. It is not the same as “light therapy”.) Chromotherapy is the act of shining a light on the body to provide a benefit. Chromotherapy may work in part because different organs and cells in the body contain chromophins and porphyrins which are activated by light.

Chromotherapy can work by three types of mechanisms. One is the light directly affects the organs because they have porphyrins or other chromophores which the light activates. One example is shining blue light on a jaundiced baby whereby the blue light directly converts the unconjugated bilirubin in the skin to conjugated bilirubin which can be excreted. The second mechanism is indirect whereby the colored light activates nerves which then transmit a signal. One example is blue light. People who use TV, computers, and cell phones before bed often have trouble sleeping because the blue light activates nerves in the eye which transmit a signal to the pineal gland and decreases melatonin production which is needed for sleep. Melatonin is an important hormone which helps to regulate the sleep-wake cycle. The third way is a colored light can activate neurons in the retina, which transmit a signal to the visual cortex. Thus, the observance for instance of red light can lead to a cheerier mood.

Chromotherapy is also called photobiomodulation because light can activate biological systems. One example to explain chromotherapy is at the cellular level. Light can activate mitochondria. Mitochondria are the powerhouse of cells and make energy in the form of ATP, adenosine triphosphate. Light activates mitochondria to produce a reactive oxygen species (ROS), which causes IkB kinase to increase NF-kB translocation to the cell nucleus, activating dozens of genes. Thus, the mechanism of action involves photon absorption in the mitochondria (cytochrome c oxidase), and ion channels in cells leading to activation of signaling pathways, up-regulation of transcription factors, and increased expression of protective genes.

Chromotherapy and its colors are being investigated in the following ways,

Red: Red is believed to increase the pulse, raise blood pressure and increase the rate of breathing. Red would be applied to support circulatory and nervous functions. It can also help with wound healing and kill some forms of cancer.

Strong Pink: Strong pink acts as a cleanser, strengthening veins and arteries.

Orange: Orange is a mixture of red and yellow. Activates and eliminates localized fat. It assists with asthma and bronchitis.

Yellow: Yellow has been used to purify the skin, help with indigestion, and strengthen the nervous system.

Green: Green, a color associated with harmony, provides a neutral, positive calming effect.

Strong Green: Strong green provides anti-infectious, anti-septic and regenerative stimulation.

Strong Blue: Strong blue lubricates joints, helps reduce stress, nervous tension and infections.

Blue: Blue promotes relaxation and calm. Blue exhibits tranquilizing qualities often used to relieve headaches and migraines, colds, stress, nervous tension, rheumatism, stomach pains, muscle cramps and liver disorders. Blue is thought to have a positive effect on all kinds of pain.

Indigo: Indigo is used to address conditions involving the eyes, ears and nose. It has a calming, sedative effect.

Violet: Violet is used to calm the nervous system, soothe organs and relax muscles. Violet has meditative qualities and is often used to treat conditions of the lymphatic system and spleen, as well as urinary disorders and psychosis.

Light therapy is currently used for many situations: red light boosts blood flow and collagen; blue can calm and tighten skin; green light for pigmentation, fine lines and anti-aging; yellow light to reduce redness and smooth skin; purple light for an allover relaxing boost; light blue to soothe; and white to improve skin color.

The FDA has approved light therapy for the following conditions:

Acne: Blue light can kill acne-causing bacteria known as Cutibacterium acnes. The combination of red and blue light also brings down redness and inflammation, and works just as well on blackheads as it does for cystic acne

Psoriasis: Blue light therapy is used to treat psoriasis.

Anti-aging (collagen production & wrinkle reduction): Experts believe that red light therapy acts on cells in the skin known as fibroblasts, which play a role in production of collagen, a protein that makes up a large part of connective tissue and helps the skin to recover when it's harmed.

Hair loss prevention and hair regrowth: The FDA has approved light therapy to treat hair loss and hair regrowth.

Pain relief: Red light therapy has been approved by the FDA for various indications such as arthritis, muscle spasms, tissue tension, joint and muscle stiffness and chronic pain.

Fat loss: Red light therapy is used for fat loss and body contouring.

Areas of current research and clinical trials with light therapy also include treatment of Alzheimer's Disease, Parkinson's Disease, traumatic brain injury (TBI), stroke, heart attack, and spinal cord injuries and nerve regeneration.

Colored automobile glass windows can make a person feel a certain way and make a statement and generate feelings of people viewing the vehicle.

Red makes a person feel passionate and energized. Red is the warmest and most dynamic of the colors—it triggers opposing emotions. It is often associated with passion and love as well as anger and danger. It can increase a person's heart rate and make them excited. If one wants to draw attention, they should use red.

Orange makes a person feel energized and enthusiastic. Orange enhances a feeling of vitality and happiness. Like red, it draws attention and shows movement but is not as overpowering. It is aggressive but balanced—it portrays energy yet can be inviting and friendly. Orange is great for a call to action to buy or subscribe to a product.

Yellow makes one feel happy and spontaneous. Yellow is perhaps the most energetic of the warm colors. It is associated with laughter, hope and sunshine. Accents of yellow help give designs energy and will make the viewer feel optimistic and cheerful. In design, it is often used to grab attention in an energetic and comforting way.

Green makes a person feel optimistic and refreshed. Green symbolizes health, new beginnings and wealth. Green is the easiest on the eyes and should be used to relax and create. Green can also feel calming and relaxing.

Blue makes a person feel safe and relaxed. Blue evokes feelings of calmness and spirituality as well as security and trust. Seeing the color blue causes the body to create chemicals that are calming. It is no surprise that it is the most favored of the colors. Dark blues can be used for corporate vehicles because it helps give a professional feel, but using too much can create a cold, disengaged feeling. Light blues give a more relaxing, friendly feel.

Purple makes a person feel creative. Purple is associated with mystery, creativity, royalty and wealth. Lighter shades of purple are often used to soothe or calm a viewer. Incorporate purple to make a design look more luxurious and wealthier or a lighter purple to show romance and mystery.

Pink makes you feel playful and romantic. Pink represents femininity and romance, sensitivity and tenderness. It is inherently sweet, cute and charming.

Brown makes a person feel down to earth. Brown creates a sense of stability and support. It is warm and friendly, practical and dependable, and can also represent the old fashioned and well established.

Gray feels serious and professional. Gray is a more mature, responsible color. Its positive connotations include formality and dependability, while the negative side can mean being overly conservative, conventional and lacking in emotion. It is safe and quite subdued, serious and reserved.

Blue light is good for depression; green light is good to alleviate anxiety; yellow, orange, and red light are good to improve mood.

Colors and color therapy can be used to treat disease. Some areas of research include:

Green is a basic color of all in healing. It is the color which is always used first and last. No matter what the ailment, one starts with green. Green is used in treating seasonal affective disorder (SAD), eye diseases, and diabetes.

Yellow is the color which signifies wisdom. Any mental deficiency can be relieved by the use of yellow. Yellow has been found very valuable for all cases of nervous conditions and for all cases where there is some mental blockage. It stimulates behavior, and can treat liver disease, gall bladder disease, and disorders of the stomach and intestine.

Pink has a tranquilizing and calming effect. It suppresses hostile, aggressive and anxious behavior.

Red is effective in healing wounds.

Blue is treatment of rheumatoid arthritis. Exposure to blue light causes significant pain relief and treatment of a wide variety of psychological problems, including addictions, eating disorders and depression. It can be used in healing injured tissue and preventing scar tissue, as well as for burns and lung conditions.

A person can make a statement with a certain colored window: black=powerful, silver=innovation and modernity, red=bold, attention seeking, blue=stability and safety, yellow=happy person, gray=does not want to stand out.

The invention allows for the automotive glass colors to be interchanged depending on the person's mood of the day or moment. Therefore, throughout the year, month, week, or day or moment, windows of different colors can be interchanged depending on a person's mood, or the weather conditions. For instance, in the northern hemisphere during winter the need to block UV is reduced while the need to add a cheerful color to prevent depression is increased. This would be opposite in the summer months when a person's mood is more upbeat because of increased sunlight while the negative effects of UV radiation both to the person and the automobile are increased by the increased light.

The present invention provides for a method of providing health benefits to an individual, by the individual being inside or outside a vehicle 10 with at least one colored automotive glass window 12 operatively attached thereto, and the at least one colored automotive glass window 12 providing health benefits to the individual. This method can include affecting a mood of the individual outside the automobile looking at the colored automotive glass window and evoking feelings of happiness, optimism, calm, or energy. This method can also include affecting a mood of the individual inside the automobile looking out the colored glass windows, often evoking feelings of happiness, optimism, calm, or energy.

The colored automotive glass can be in any shape or size to fit any part of a vehicle, such as windshields, side windows (shown in FIGS. 2-4), back windows, moonroofs or sunroofs.

Colored automobile windows can be made in different ways. Glass can be colored: 1) while it's being made and it's in the molten state by adding metals or other chemicals or a combination thereof, 2) by making a laminated glass with a colored interlayer, or 3) by adding a colored film layer film on top of the glass.

The colored automotive glass can be made by several methods known in the art such as one method by adding a metal compound or metal powder oxides or sulfides to glass while molten, forming a specific glass shape, and then cooling the molten glass, providing a colored automotive glass. Various metals and the colors they produce are listed in TABLE 2.

TABLE 2
Metals Used to Impart Color to Glass

| | |
|---|---|
| Cadmium Sulfide | Yellow |
| Gold Chloride | Red |
| Cobalt Oxide | Blue-Violet |
| Manganese Dioxide | Purple |
| Nickel Oxide | Violet |
| Sulfur | Yellow-Amber |
| Chromic Oxide | Emerald Green |
| Uranium Oxide | Fluorescent Yellow, Green |
| Iron Oxide | Greens and Browns |
| Selenium Oxide | Reds |
| Carbon Oxides | Amber-Brown |
| Antimony Oxides | White |
| Copper Compounds | Blue, Green, Red |
| Tin Compounds | White |
| Lead Compounds | Yellow |

Combinations of metals can be used to achieve colors. For instance, iron(II) oxide can be used with chromium to produce a rich green. Sulfur with carbon and iron salts produces amber colors from yellow to black. Sulfur and calcium produce a deep yellow. Manganese can be added to iron to remove green tints or produce an amethyst color. Manganese dioxide can be used to remove green colors. Chromium with tin oxide and arsenic provides emerald green colors. Cadmium with sulfur provides deep yellow, and cadmium with selenium and sulfur provides bright red and orange. Titanium can be used with other compounds to intensify and brighten other colors.

There can be other ways to color glass using different metals and chemicals other than metals.

Another method, and the preferred embodiment is using a colored interlayer in laminated glass.

A preferred embodiment is poly vinyl butyrate (PVB) as a colored interlayer between sheets of glass to make laminated glass as described below.

Another method is adding a colored film layer to the outside of the glass.

There are two main types of automotive glass: tempered and laminated. Tempered glass is a specific make while laminated glass is the “conjoining” of two pieces of glass either tempered, annealed, heat strengthened, chemically strengthened or other types of glass which can be done in combination, e.g., tempered conjoined to annealed glass, with a colored inner layer of PVB between the two glass sheets to hold them together.

The preferred embodiment is colored laminated glass because of the safety feature that it doesn't shatter but any colored glass can be used. Colored laminated glass gets it color from the film interlayer and thousands of different colors can made using the interlayer.

Laminated glass has many uses including furniture, windows, walls, and doors because of its safety features. Laminated glass is typically formed by using two pieces of glass (see types of glass below) and an interlayer, a polyvinyl butyral (PVB) interlayer is the preferred embodiment. Other types of polyvinyl interlayer can also be used such as, but not limited to, polyvinylchloride (PC), polycarbonate, ethylene vinyl acetate (EVA), polyethylene terephthalate (PET), polyurethane, polyester, polyethylene naphtholate, polycarbonate, polymethylmethacrylate, polyether Sulfone, nylon, polyarylate, and cycloolefin polymer, acoustic polyvinyl butyral (A-PVB), plasticized polyvinyl chloride (PVC), or thermoplastic polyurethane (TPU), or a combination; and combinations are useful when adding or enhancing a characteristic of the laminated glass like preventing infra-red sun rays from penetrating the glass by using a reflective interlayer.

The PVB used in “standard” automotive windows is clear and it has virtually always been clear in the currently made automotive glass windows because automotive windows are clear; while the invention makes colored laminated glass. This makes intuitive sense that current car windows are clear since car windows are made to see out of, and clear glass is presumed to be the best for an automotive window. The typical colored laminated glass is used in buildings or furniture or walls where the colored glass adds aesthetics and prevents an “outside” person from seeing in. Although counterintuitive, the invention demonstrates that colored glass does have properties that allows light transmission so a person inside the car can see outside while the colored glass also provides health and safety benefits. Thus, colored glass performs the functions of clear glass, visibility to the outside world, and also provides health benefits as described herein.

Colored automobile laminated glass windows can also be crafted for better attenuation of acoustic noise, reduction of UV and/or IR light transmission, depending on the interlayer used, while maintaining its health benefits.

Laminated colored glass windows are generally produced in several steps: 1) first a mold is made, 2) the glass is made using the mold and can be bent if required, 3) the PVB is placed between two sheets of clear glass and the sandwich structure is heated as nip rollers squeeze the sheets in contact with the softened PVB to initially bond the glass plates together, and 4) autoclaving of the heated glass is used to complete the bonding process. Depending on the process and manufacturer some steps may not be used, additional steps may be added, and modifications of these steps may be made.

The key feature of laminated glass is the minimization of dispersion of glass fragments if the glass is broken along with thermal stability and weatherability because the interlayer holds the glass together preventing fragmentation.

The preferred PVB in the present invention is a film including approximately 75% PVB resin and 25% plasticizer termed SAFLEX® (Eastman). SAFLEX® are plasticized with SANTICIZER® (Valtris Specialty Chemicals) S2075 plasticizer, or triethylene glycol di-2-ethyl hexanoate. Specialty grades of SAFLEX® have different compositions. Small quantities of other materials can be added for adhesion control, ultra-violet screening agents and color. It can also have different compositions. This preferred PVB system provides thousands of color options with different characteristics of light transmission, UV light protection, heat and sound protection with a typical thickness between 0.38 millimeters (0.015 inches) to 1.52 millimeters (0.060 inches). If a thickness of the interlayer film is thinner than 0.3 millimeters (mm), a handling of the film is difficult. On the other hand, if the thickness is thicker than 2 mm, an outer appearance defect may occur due to a degassing failure at a time of a lamination process. Hence, the thickness desirably falls within a range of 0.3 millimeters through 2 millimeters, but smaller and larger sizes can be used depending on the characteristics of the interlayer.

There are five main types of glass (annealed glass, heat-strengthened glass, chemically strengthened glass, fully tempered glass, and laminated glass), which are associated with various glass strengths for load and impact. Laminated glass can be made from using two sheets of any combination of annealed glass, heat strengthened glass, fully tempered glass, chemically strengthened glass or any other type of glass.

Annealed glass is the base glass that is formed from the float glass process. As the molten glass begins, under a controlled manner, to cool, it hardens. The slow cooling allows a very limited amount of stress to be retained in the glass and thus the glass can be cut along intentional score marks. Annealed glass typically has excellent optics and minimal optical distortion. Glass can be ultra-clear, clear or colored. It can be smooth, patterned or have wire set into it. Annealed glass can be readily cut to size and shapes as needed, edged, coated with metal coatings, ceramic frit, paint and inks and laminated. Annealed glass is used as the base product to form other fabricated glass.

Heat Strengthened Glass is also known as semi-tempered or semi-toughened glass. In general, the heat strengthening process involves first cutting annealed glass to the desired size and shape, edging the glass as indicated, then heating the annealed glass back up to near 650° C. (1200° F.) and then cooling it rapidly. This rapid cooling causes stress in the glass and forms a compressive laver on the glass surfaces and a tension layer in the core. The heat strengthening process increases the mechanical and thermal strength of annealed glass, making it historically twice as strong as annealed glass. Heat-strengthened glass tends to have low levels of glass distortion and bow caused by the strengthening process. Upon breaking, the pattern is similar to annealed glass. Except for cutting after strengthening (with traditional methods) heat-strengthened glass can be fabricated with the same value add coatings and lamination as annealed glass. Heat-strengthened glass is typically used in areas that benefit from low optical distortion, need to avoid the potential of spontaneous breakage, need increased mechanical strength versus annealed glass, or are subjected to thermal stress from temperature gradients, and laminated glass applications requiring larger break patterns such as hurricane, balcony (undrilled) and blast and glass shard retention post-breakage.

Tempered glass is also known as toughened glass. In general, the tempering process involves first cutting annealed glass to the desired size and shape, edging the glass as indicated, then heating the annealed glass back up to near 650° C. (1200° F.) and then very rapidly cooling it. This high speed of cooling causes stress in the glass and forms a compressive layer on the glass surfaces and a tension layer in the core. The delineation of zones in the tempered glass is similar to heat-strengthening but the compressive strength of the surface is significantly higher. The tempering process increases the mechanical and thermal strength of annealed glass, making it historically four times as strong as annealed glass. Tempered glass may have glass roller wave distortion and bow caused by the strengthening process. Upon breaking, the pattern is unique in that it forms small cube-like pieces which may or may not fall out at the time of impact but have virtually no residual strength as a glass unit when faced with subsequent load. Tempered glass can be fabricated with the same value add coatings and lamination as annealed and heat-strengthened glass. Tempered glass is typically used in areas that need increased mechanical strength versus annealed and heat-strengthened glass, drilled (prior to tempering) or used in point support systems, are subjected to thermal stress from temperature gradients, locations requiring safety glazing without glass fall-out concerns, laminated glass applications requiring added glass strength and glass shard retention post breakage.

Chemically strengthened glass is annealed glass is strengthened by a chemical exchange of ions. The chemically strengthening process involves first cutting annealed glass to the desired size and shape, edging the glass as indicated, then submerging the glass in a solution that promotes the ion exchange where the smaller ions are replaced by larger ions. This exchange causes a compressive layer on the glass surfaces and a tension layer in the core. The strengthening process increases the mechanical and thermal strength of the glass, however there is no common value used in the industry to account for the strength enhancement. Chemically strengthened glass tends to have levels of glass distortion and bow similar to the annealed base glass. Upon breaking, the pattern is similar to annealed glass, but in some cases the break pattern can be adjusted to be smaller. Cutting after strengthening (with traditional methods) may weaken the glass. Fabrication of chemically strengthened glass is beginning to be more common in the industry. Lamination of chemically strengthened glass is possible, however compatibility studies should be undertaken prior to use. Chemically strengthened glass is typically used in areas that benefit from low optical distortion, need to avoid the potential of spontaneous breakage, or need increased mechanical strength versus annealed glass, and laminated glass applications requiring added strength, low distortion and glass shard retention post-breakage.

Any of the above types of glass can be laminated. The glass thickness and type are typically specified based on the performance desired for the glazing. Common safety glazing can be produced with thin glass plies (˜3 mm (0.125 inch) and in general, an interlayer 0.76 mm (0.030 inch) to 1.60 mm. SAFLEX® or VANCEVA® (Eastman) polyvinyl butyral (PVB) is preferred in this embodiment. Laminated glass offers many advantages over monolithic glass types. Safety and security are historically the best-known characteristics of laminated glass as the interlayer tends to retain the broken glass shards and act as a penetration barrier. This aids in reducing the likelihood of falling glass on pedestrians and can deter entry through the glass for some time depending on the composition of the laminated glass and glazing system. Today, laminated glass can bring more than just safety and impact resistance: noise damping, windborne debris and blast resistance, structural capacity, enhanced UV radiation screening, solar control and an array of thousands of color options are all available in laminated glass configurations depending on the interlayer.

The preferred embodiment is to use laminated glass. The PVB layer provides color and clarity. All colors can be synthesized from the basic 8 colors of the rainbow. And the darkness of each color can be varied which can increase or decrease light transmission depending on the position of the colored glass window; windshield and front and back side windows and a back window have different state and country regulations in regard to light transmission. Thus, laminated glass with a colored PVB inner layer provides thousands of different color/transparencies with different UV and heat reflectance.

Colored glass windows can also be done with other types of glass like tempered glass and heat and chemically strengthened glass by adding different metals and chemicals to the glass during the manufacturing process.

Colored glass windows can also be made by applying a colored film to the window.

SAFLEX® brand PVB is a film consisting of approximately 75% PVB resin and 25% plasticizer. Specialty grades of SAFLEX® have different compositions. Small quantities of other proprietary materials are added for adhesion control, ultra-violet screening agents and color. For laminated glass designed with VANCEVA® interlayers, there can be many types of style, color, glass, and translucency. VANCEVA® colors interlayer system, a collection of 16 foundational and vibrant colors that can be combined to obtain thousands of transparent, translucent or solid options. The VANCEVA® color interlayer system consists of eight foundation colors, two translucent white colors, four specialty colors, and solid white and black colors. Of these interlayers, any one, two, three or four can be combined to create a piece of colored laminated glass—combining color interlayers to produce thousands of transparent, translucent, or solid colors that create just the right look, safety, health benefit, and ambiance.

SAFLEX® has all colors which meet the requirements for light transmission of any state regulations from <35%, 35%-49%, 50%-69%, or 70% and greater light transmissions.

Laminated glass made with VANCEVA® color interlayers can be made to deliver effective protection from harmful UV radiation, glare, and solar energy transmittance and reduces solar heat gain-blocking up to 99% of damaging UV light up to 380 nm to help retard color fading and the deterioration of fabrics and furnishings.

Laminated glass consists of a tough protective interlayer made of polyvinyl butyral (PVB) bonded together between two sheets of glass under heat and pressure. Once sealed together, the glass “sandwich” behaves as a single unit and looks like normal glass. Annealed, semi-toughened, strengthened, or tempered glass can be used to produce laminated glass. Laminated glass provides durability, high-performance, and multifunctional benefits while preserving the aesthetic appearance of the glass. When impacted, laminated architectural glass will typically remain intact and may help prevent further damage caused by shattered glass or protect the automobile from “run-by” thefts where thieves attempt to quickly break a window and grab something inside the car like a box or briefcase.

Glass laminates can be substantially flat or shaped for certain applications. For example, glass laminates can be formed as bent or shaped parts for use as windshields and side and back automobile windows. The structure of a shaped glass laminate can also be simple or complex. In certain embodiments, a shaped glass laminate can have a complex curvature where the glass sheets have a distinct radius of curvature in two independent directions. Such shaped glass sheets can thus be characterized as having “cross curvature,” where the glass is curved along an axis parallel to a given dimension and also curved along an axis perpendicular to the same dimension. An automobile sunroof can be an example.

Shaped glass laminates according to certain embodiments can be defined by a bend factor, where the bend factor for a given part is substantially equal to the radius of curvature along a given axis divided by the length of that axis.

SAFLEX® Color, the conventional PVB interlayer can be used for:

Safety: in case of glass breakage from human or accidental impact, the shards of broken glass remain adhered to the interlayer providing a reduction in cutting or piercing injuries. With properly glazed laminated glass in a framing system, fall through protection can also be provided.

Storms: laminated glass with adequate interlayer thickness is capable of thwarting wind-borne debris from penetrating the façade and subsequently allowing destructive internal pressurization in severe windstorms.

Security: a laminated glass configuration can be incorporated into a system to provide a sufficient level of protection against man-made intentional attacks, including vandalism and burglary, explosion resistance and ballistic assault.

SAFLEX® Acoustic can also be used and is an engineered tri-layer interlayer that provides enhanced sound damping between 1000-4000 Hertz (Hz) as compared to conventional PVB interlayer. The enhanced performance allows the glazing construction to be designed using less air space and/or thinner glass to achieve the same sound reduction rating versus conventional and historic practices.

SAFLEX® Solar can also be used and is a solar absorbing PVB interlayer, brings improved solar control by reducing solar heat gain versus clear and conventional laminated glass without coatings. Saflex Solar can also be incorporated into single unit laminated glass with low-emissivity hard coatings or into insulating glass units (IGU) with or without low-emissivity glasses.

The present invention provides for a method of providing a colored automotive glass window in a vehicle, by making a colored automotive glass window by 1) adding a metal compound or other chemical to molten glass during the manufacturing process forming colored glass, 2) by bonding multiple layers of glass together while under pressure and heat, with a colored interlayer to make colored laminated glass, or 3) by layering a colored plastic, chemical, or compound on top of a clear glass window, and installing the colored automotive glass window in the vehicle. This automotive vehicle can be driven on public roads and highways because the laminated colored glass or other colored glass meets safety requirements.

The present invention shows that colored glass windows 12 provide good visual acuity, health benefits, and safety and with online automobile ordering increasing, inventory issues are solved as there would not be a need to drastically increase inventory since customers can pick the colored glass window at the time of ordering.

The present invention demonstrates that colored windows with light transmission from 0% to 99% for vehicles can have an advantageous purpose relating to health benefits for the person(s) inside the car, and person(s) outside the car, by affecting the mental health and well-being of a person, particularly since a person spends so much time in their car or a lot of time looking at cars on public roads and highways. A person's mood and mental health can be affected by spending hundreds of hours a year in a car and this invention uses colored glass windows to positively help a person's health and well-being and mental health. Colored glass windows can also protect the interior of the car from the effects of UV light and the colored glass windows can reflect heat and sound, also protecting the interior of the car (health of the car) and can also save on fuel consumption. As described above, the benefits of colored glass automotive windows for health are from: 1) the psychological effects of colored windows on mood, 2) the health benefits of chromotherapy (photobiomodulation), the shining of colored light on the body, 3) the health benefit of reduced exposure to UV light on persons in the car, 4) prevention of weathering effects on the interior of the car from heat and UV light leading to better “health” of the automobile. Furthermore, the colored glass windows 12 meet safety standards for automobile windows such as DOT testing. By adding a colored interlayer to laminated glass, the colored glass maintains DOT testing certification.

The invention is further described in detail by reference to the following experimental examples. These examples are provided for the purpose of illustration only and are not intended to be limiting unless otherwise specified. Thus, the invention should in no way be construed as being limited to the following examples, but rather, should be construed to encompass any and all variations which become evident as a result of the teaching provided herein.

### Example 1

In the present invention, the back side glass windows 12 (both sides) of a 2019 Mazda 3 vehicle 10 were replaced. The laminated clear bent tempered glass window was removed, and a mold was made which was used as the template to generate an orange color bent laminated glass window using SAFLEX #0545 as an interlayer between two annealed glass plates. The orange glass laminated window was thicker than the original clear tempered glass window and therefore the inner rear car door window frame needed to be modified so the thicker laminated window could fit in the window frame so as to allow a functioning window which can roll up and down to close and open.

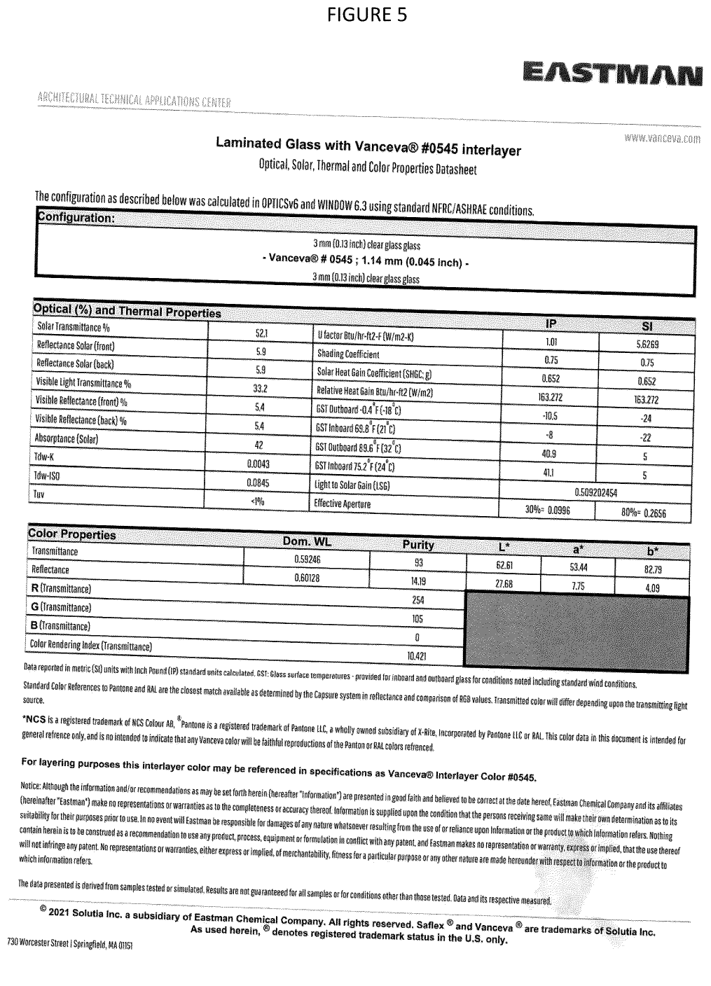

More specifically, FIG. 2 shows that a colored side window 12 installed in a vehicle (car) 10 and driven on public roads, 2019 Mazda 3, for normal everyday use. The color is orange which comes from the inner layer PVB (VANCEVA®#0545 interlayer, see FIG. 5 for specifications), and produces orange light into the interior of car when the car is in sunlight (see FIGS. 3-4). In addition, the person sitting in the car can see to the outside (see FIGS. 3-4). The light transmission meets the Nevada state regulations where the automobile is registered. However, to install an orange colored window 12 for a 2019 Mazda 3, problems needed to be solved. One problem was that in many cars made in 2019, side front and back automobile windows are made from tempered glass. Tempered glass is typically stronger than laminated glass and therefore is thinner, and in this case the laminated glass was thicker, (although eventually this problem can be solved since laminated glass made with sheets of tempered glass can be thinner and stronger). The average car side windows made with tempered glass are <5 mm in thickness while the laminated orange colored back side window 12 (in FIGS. 2-4) is about 6.92 mm in thickness; 2.7 mm thickness for each glass panel and 1.52 mm thickness for the orange PVB inner layer (VANCEVA® #0545). The 6.92 mm thick laminated orange glass did not fit into the window frame of the 2019 Mazda 3 which was made of tempered glass window. The window frame needed to be adjusted. However, there are new methods to make thinner laminated glass using two sheets of relatively thin chemically strengthened glass, such as CORNING®. This is being developed (such as in U.S. patent application Ser. No. 16/788,363 filed Jun. 11, 2020.)

The VANCEVA® PVB interlayer is made of four plies which are approximately 0.38 mm thick. Each number ply, 0-5-4-5 corresponds to a specific color and order that the sheets need to be aligned to obtain the specified orange color.

The interlayer sheets #0, #4, #5 that constitute the orange PVB interlayer were purchased from VANCEVA® (SAFLEX®) corresponding to color number 0545; there are four plies layered according to the number sequence. It produces an orange color with 33.2 light transmission, 52.1 solar transmission, a solar absorbance of 42, solar heat gain coefficient 0.65, shading coefficient 0.75, U factor (BTU/hr-ft-2) 1.01 (see FIG. 5). The orange laminated glass window was installed in the rear car windows since light transmission is 33.2% meeting the Nevada code for light transmission in side rear windows. Nevada is where the vehicle is registered.

In some states, the rear windows need at least 35% light transmission and then orange SAFLEX® number 0458, using #0, #4, #5, #8, to make the four plies, can be used to make the interlayer and the orange colored window with 43.5% light transmission can be used. In some states the side front windows require at least 50% light transmission and other states 70% light transmission and orange SAFLEX® number 0141 and 0018 can be used with 59.9% and 73.99% light transmission, respectively.

The orange glass window can be shown to pass DOT, the safety testing for car glass windows thus showing that coloring the window with a colored interlayer, orange, in place of a clear interlayer does not affect safety testing of the glass window.

Throughout this application, various publications, including United States patents, are referenced by author and year and patents by number. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology, which has been used is intended to be in the nature of words of description rather than of limitation.

Obviously, many modifications and variations of the present invention are possible in light of the above teachings. It is, therefore, to be understood that within the scope of the appended claims, the invention can be practiced otherwise than as specifically described.

What is claimed is:

1. A vehicle comprising at least one colored automotive glass window operatively attached to said vehicle.

2. The vehicle of claim 1, wherein said at least one colored automotive glass window is made of a material chosen from the group consisting of annealed glass, chemically strengthened glass, heat strengthened glass, tempered glass, and laminated glass.

3. The vehicle of claim 1, wherein color in said at least one colored automotive glass window is produced when manufactured using metal or chemicals.

4. The vehicle of claim 1, wherein color in said at least one colored automotive glass window is produced by applying a color film to an outside of clear glass.

5. The vehicle of claim 1, wherein color in said at least one colored automotive glass window is produced by using laminated glass with a colored interlayer.

6. The vehicle of claim 5, wherein said colored interlayer is made of a material chosen from the group consisting of poly vinyl butyrate (PVB), polyvinylchloride (PC), polycarbonate, ethylene vinyl acetate (EVA), polyethylene terephthalate (PET), polyurethane, polyester, polyethylene naphtholate, polycarbonate, polymethylmethacrylate, polyether Sulfone, nylon, polyarylate, and cycloolefin polymer, acoustic polyvinyl butyral (A-PVB), plasticized polyvinyl chloride (PVC), thermoplastic polyurethane (TPU), and combinations thereof.

7. The vehicle of claim 1, wherein color in said at least one colored automotive glass window is chosen from the group consisting of red, orange, yellow, green, blue, indigo, violet, and combinations thereof.

8. The vehicle of claim 7, wherein said at least one colored automotive glass window includes at least two colors.

9. The vehicle of claim 1, wherein said at least one colored automotive glass window has light transmission from 0 to 99%.

10. The vehicle of claim 1, wherein said at least one colored automotive glass window meets U.S. state or country requirements for light transmission.

11. The vehicle of claim 1, wherein said at least one colored automotive glass window meets all automotive safety testing required by U.S. state or country wherein said vehicle is registered.

12. The vehicle of claim 1, wherein said at least one colored automotive glass window passes DOT testing for safety.

13. The vehicle of claim 1, wherein said at least one colored automotive glass window blocks an item chosen from the group consisting of UV light, light (heat), sound, and combinations thereof.

14. The vehicle of claim 1, wherein said at least one colored automotive glass window is changeable in said vehicle to a second colored automotive glass window in a different color.

15. The vehicle of claim 1, wherein said at least one colored automotive glass window is sized to fit a window chosen from the group consisting of a windshield, a side window, a back window, a moonroof, and a sunroof.

16. A method of providing colored automotive glass windows in a vehicle, including the steps of:
making a colored automotive glass window by a method chosen from the group consisting of 1) adding a metal compound or other chemical to molten glass during the manufacturing process forming colored glass, 2) by bonding multiple layers of glass together while under pressure and heat, with a colored interlayer to make colored laminated glass, and 3) by layering a colored plastic, chemical, or compound on top of a clear glass window; and
installing the colored automotive glass window in a vehicle.

17. The method of claim 16, wherein color in the colored automotive glass window is chosen from the group consisting of red, orange, yellow, green, blue, indigo, violet, and combinations thereof.

18. The method of claim 16, wherein the colored interlayer is made of a material chosen from the group consisting of poly vinyl butyrate (PVB), polyvinylchloride (PC), polycarbonate, ethylene vinyl acetate (EVA), polyethylene terephthalate (PET), polyurethane, polyester, polyethylene naphtholate, polycarbonate, polymethylmethacrylate, polyether Sulfone, nylon, polyarylate, and cycloolefin polymer, acoustic polyvinyl butyral (A-PVB), plasticized polyvinyl chloride (PVC), thermoplastic polyurethane (TPU), and combinations thereof.

19. A method of providing health benefits to an individual, including the steps of:
the individual being inside or outside a vehicle with at least one colored automotive glass window operatively attached thereto; and
the at least one colored automotive glass window providing health benefits to the individual.

20. The method of claim 19, wherein said providing health benefits step is further defined as affecting a mood of the individual outside the automobile looking at the colored automotive glass window and evoking feelings of happiness, optimism, calm, or energy.

21. The method of claim 19, wherein said providing health benefits step is further defined as affecting a mood of the individual inside the automobile looking out the colored glass windows, and evoking feelings of happiness, optimism, calm, or energy.

22. The method of claim 19, wherein color in the at least one colored automotive glass window is chosen from the group consisting of red, orange, yellow, green, blue, indigo, violet, and combinations thereof.

23. The method of claim 19, further including the step of changing the colored automotive glass window to a second colored automotive glass window in a different color based on the individual's mood or weather conditions.

24. The method of claim 19, wherein said providing health benefits step is further defined as providing chromotherapy (photobiomodulation) to the individual inside the vehicle.

25. The method of claim 19, wherein said providing health benefits step is further defined as treating a disease chosen from the group consisting of arthritis, muscle spasms, tissue tension, joint and muscle stiffness, or chronic pain.

26. The method of claim 19, wherein said providing health benefits step is further defined as reducing the individual's exposure to UV light and/or sound.

27. The method of claim 19, wherein said providing health benefits step is further defined as reducing the individual's stress.
